(19) United States
(12) Patent Application Publication
(10) Pub. No.: US 2007/0155955 A1
(43) Pub. Date: Jul. 5, 2007

(54) Amylin aggregation inhibitors and use thereof

(75) Inventors: Youcef Fezoui, Chene-Bourg (CH); Claudio Soto-Jara, Friendswood, TX (US)

Correspondence Address: OBLON, SPIVAK, MCCLELLAND, MAIER & NEUSTADT, P.C., 1940 DUKE STREET, ALEXANDRIA, VA, 22314, US

(73) Assignee: APPLIED RESEARCH SYSTEMS ARS HOLDING N.V., CURACAO (NL)

(21) Appl. No.: 10/549,502

(22) PCT Filed: Mar. 17, 2004
(86) PCT No.: PCT/EP04/50320

§ 371 Date: Sep. 20, 2006

(30) Foreign Application Priority Data
Mar. 18, 2003 (EP) 03100683.6

(51) Int. Cl. A61K 38/08; C07K 7/06

(52) U.S. Cl. 530/329; 514/16

(57) ABSTRACT

Short peptides and derivatives or analogs thereof for the treatment or prevention of IAAP related disorders, in particular amyloid deposits associated with Type I or Type II diabetes are herein described. These peptides and/or their derivatives have been designed to inhibit the formation of amylin fibrils which are implicated in the pathogenesis of diabetes Type II as well as to dissolve the fibrillar deposits already formed

### FIELD OF THE INVENTION

The invention relates to the field of β-sheet breaking peptides, particularly to their use in the treatment of Type II diabetes.

### BACKGROUND OF THE INVENTION

Type-II diabetes is a heterogeneous and multi-factorial disease characterized by beta-cell failure, insulin resistance, and the presence of amyloid deposits in the pancreatic islets of about 90% of patients (Anguinao et al., 2002). The major component of these deposits is a 37 amino acid peptide called islet amyloid polypeptide (IAPP) or amylin (Cooper et al., 1987).

Amylin is a normal secretory product of the pancreatic beta-cells, stored in the same granules as insulin and is co-released with insulin during the process of exocytosis. The normal function of soluble amylin is to control glucose homeostasis, possibly as an insulin counter-regulatory hormone (Rink et al., 1993). However, under certain conditions, the increase in amylin secretion leads to formation amyloid fibrils that deposit in the pancreas of Type II diabetes patients. The formation of amyloid deposits is strongly associated with continuous decline of beta-cell function and the progression of the disease (Höppener et al., 2000). A strong evidence for the importance of amyloid in the pathogenesis of diabetes Type II comes from genetic studies showing that mutations in amylin gene result in early onset hereditary disease, specially when accompanied with obesity.

IAPP fibril formation has also been found in patients with Type I diabetes post-transplantation. Therefore, the amyloidogenic protein IAPP, that has been shown to induce β-islet cell toxicity in vitro, may contribute to the loss of β-islet cells (Langhorans) and organ dysfunction by the formation of fibrils in the pancreas of Type I or Type II diabetic patients.

Amylin fibril formation involves the conversion of an irregularly structured conformer (random coil, soluble form) into highly stable β-sheet-rich fibrillar aggregates. As in the case of amyloid beta in Alzheimer's disease, the random-coil to β-sheet conversion is believed to be one of the earliest events in amylin fibril formation. Therefore, diabetes Type-II is included in the group of diseases in which protein misfolding and aggregation is a hallmark event in the disease (Soto et al., 2000; Carrell et al., 1997 and Dobson, 1999). The central region of amylin sequence, residues 20-29, has boon found to be very important for fibril formation (Glenner et al, 1988 and Westernark et al., 1990) and thus might be the sequence where the conversion to β-sheet occurs.

Some fragments of native amylin have been developed as modulators of IAPP aggregation (EP 885904 and US 2002/0119926). In addition, some analogues of amylin have been developed as amylin agonists for the treatment of hypoglycaemic conditions in which enhanced amylin action is of benefit (BP 1162207 and U.S. Pat. No. 6,610,824).

A human amylin analogue of 37 amino acid, Pramlintide acetate (Symlin®) is being developed by Amylin Pharmaceuticals Inc. as an adjunct with insulin for the potential prevention of complications of Type I diabetes and as a single agent for Type II diabetes.

One approach to the treatment and prevention of disorders associated with protein misfolding and aggregation has been to develop short peptides having some sequence homology to the natural protein sequence believed to be involved in amyloid formation, but also having one or more amino acids that disfavour or destabilise the formation of β-pleated sheet conformations. The peptides prevent the aggregation of β-amyloid, and thereby prevent its cytotoxic effects. This approach has been suggested in Alzheimer's disease and in prion-related disorders (WO 96/39834, New York University and WO 01/34631, Axonyx Inc.) leading to the development of the β-sheet breaking peptides shown below, amongst others:

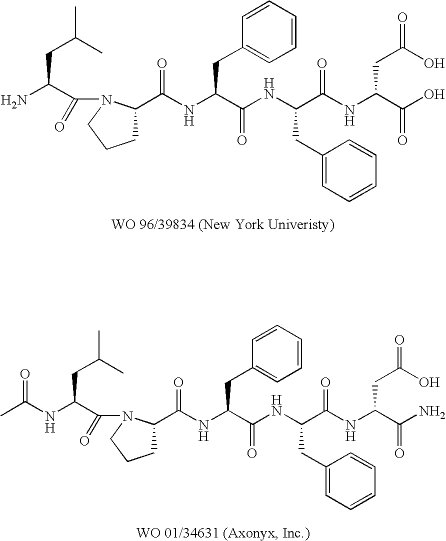

The amino acid proline has been used frequently to destabilize the formation of β-sheet structure because its physical and structural properties determine that this residue is rarely found inside β-sheet structures. Interestingly, a comparison of the amylin sequence from different species shows that species where a diabetic condition has been reported (human and cats) have a lower number of prolines in the 20-29 region of amylin compared with rodents where amyloid deposition and diabetes has not been shown (FIG. 1) (Moriarty et al., 1999). These findings are consistent with the role of amylin aggregation in the pathogenesis of diabetes Type II and further support the concept of β-sheet breaker peptides useful as inhibitors of amyloid formation

### SUMMARY OF THE INVENTION

It is an object of the invention to provide substances which are suitable for the treatment of and/or prevention of and/or delaying the progression of diabetes, notably Type II diabetes.

It is also an object of the invention to provide substances which are suitable for reducing or inhibiting amylin aggregation.

It is notably an object of the invention to provide β-sheet breaking peptides which are suitable for reducing or inhibiting amyloid formation by amylin.

In a first aspect, the invention provides peptides having an amino acid sequence of Formula I (SEQ ID NO. 1):
X₁FGAPX₂ X₃ in which
X₁, is selected from Aspartic acid and a derivative thereof;
X₂ is Leucine or when X₃ is absent, X₂ is selected from Leucine and a derivative thereof,
X₃ can be absent or is selected from Aspartic acid and a derivative thereof, wherein F represents Phenylalanine, G represents Glycine, A represents Alanine and P represents Proline.

In a second aspect the present invention provides compounds of Formula I for use as a medicament.

In a third aspect, the invention provides a pharmaceutical composition comprising a compound of Formula I, together with a pharmaceutically acceptable excipient or carrier.

In a fourth aspect, the invention provides a use of Formula I for the preparation of a medicament for the treatment and/or prevention of a diabetic condition selected from post-transplantation Type I diabetes and Type II diabetes.

In a fifth aspect the invention provides a method for treating a disease associated with abnormal folding of the islet amyloid polypeptide.

In a sixth aspect the invention provides a method for treating a patient suffering from diabetes, notably Type II diabetes.

### DETAILED DESCRIPTION OF THE INVENTION

The following paragraphs provide definitions of various chemical moieties and terms, and are intended to apply uniformly throughout the specification and claims unless an otherwise expressly set out definition provides a different definition.

The term “peptide” is ordinarily applied to a polypeptidic chain containing from 3 to 30 or more contiguous amino acids, usually from 3 to 20 contiguous amino acids. Such peptides can be generated by methods known to those skilled in the art including partial proteolytic cleavage of a larger protein, chemical synthesis, or genetic engineering.

The expression “derivative or analogue” means any compound the chemical structure of which contains modifications with respect to the parent peptide, but which maintains at least 50%, more preferably at least 75%, most preferably at least 90% of the biological activity of a compound of Formula I.

The term “derivatives” as herein used refers to derivatives which can be prepared from the functional groups present on the lateral chains of the amino acid moieties or on the N-/ or C-terminal groups according to known methods. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups.

The term “salts” herein refers to both salts of carboxyl groups and to acid addition salts of amino groups of the peptides, polypeptides, or analogs thereof, of the present invention Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

“C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like. By analogy C₁-C₅-alkyl refers to monovalent alkyl groups having 1 to 5 carbon atoms.

“C₁-C₆ Acyl” refers to a group —C(O)R where R includes H and “C₁-C₅-alkyl” groups. This term includes formyl (—C(O)H) and acetyl (—C(O)CH₃).

“Amino” refers to the group —NRR′ where each R,R′ is independently hydrogen or “C₁-C₆-alkyl”.

“Halogen” refers to fluoro, chloro, bromo and iodo atoms.

“Amidated amino acid” refers to an amino acid wherein the hydroxy group from the acid moiety has been replaced by an amino group.

“Acylated amino acid” refers to an amino acid wherein the one hydrogen atom on tie nitrogen has been replaced by a C₁-C₆ acyl group. Further, an “acetylated amino acid” refers to an amino acid wherein the one hydrogen atom on the nitrogen has been replaced by an acetyl group.

“Substituted” refers to groups substituted with from 1 to 5 substituents selected from the group comprising “C₁-C₆-alkyl”, “amino”, “halogen”, trihalomethyl, cyano, hydroxy, mercapto, nitro, and the like.

The polypeptides and the peptides of the present invention can be in other alternative forms which can be preferred according to the desired method of use and/or production, is for example as active fractions, precursors, salts, derivatives, conjugates or complexes.

Compounds of the invention are suitable for the treatment and/or prevention of a diabetic condition, including a disease associated with abnormal folding of the islet amyloid polypeptide, Type I or Type II diabetes and postransplantation Type I or Type II diabetes.

The compounds of the invention may be prepared by any well-known procedure in the art, including chemical synthesis technologies.

Examples of chemical synthesis technologies are solid phase synthesis and liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective group include tBoc (t-butoxycarbonyl), Cl-Z (2-chlorobenzyloxycarbonyl), Br-Z (2-bromobenzyloxycabonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl₂-Bzl (2,6-dichlorobenzyl) for the amino groups; NO₂ (nitro) and PmC (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The present invention provides compounds capable of controlling IAPP aggregation and fibril formation

The activity of the compound of the invention in inhibiting the amylin fibrils can be detected using, for example, an in vitro assay, such as that described by Klunk et al., 1999 and Fezou et al., 1999, which measures the ability of compounds of the invention to prevent the aggregation of the islet amyloid polypeptide (IAPP). Results are reported in the Examples.

Amylin fibrils are cytotoxic, inducing cell death by apoptosis (Lorenzo et al., 1994). Compounds of the invention can be tested for their ability to prevent cytotoxicity of amylin fibrils. Results are reported in the Examples.

In one embodiment, the invention provides compounds according to Formula I, wherein X₁ is selected from Aspartic acid and acetylated Aspartic acid.

In another embodiment, the invention provides compounds according to Formula I, wherein X₂ is selected from Leucine and amidated Leucine.

In another embodiment, the invention provides compounds according to Formula I, wherein X₃ is absent.

In another embodiment, the invention provides compounds according to Formula I, wherein X₁ is Aspartic acid and X₂ is Leucine.

In another embodiment, the invention provides compounds according to Formula I, wherein X₁ is acetylated Aspartic acid and X₂ is amidated Leucine.

In another embodiment, peptides of the invention are of following Formula II: 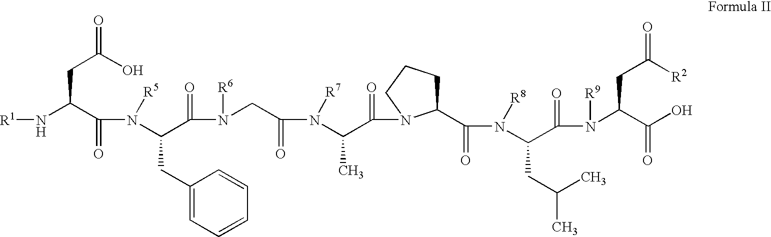
wherein R₁ is selected from H, optionally substituted C₂-C₆ acyl and optionally substituted C₁-C₆ alkyl preferably H and acetyl; R² is selected from OH and NR³R⁴, wherein R³ and R⁴ are independently selected from H and optionally substituted C₁-C₆ alkyl, preferably R² is selected from OH and NH₂; R⁵, R⁶, R⁷, R⁸ and R⁹ are independently selected from H and C₁-C₆ alkyl.

In another embodiment, peptides of the invention are of following Formula III: 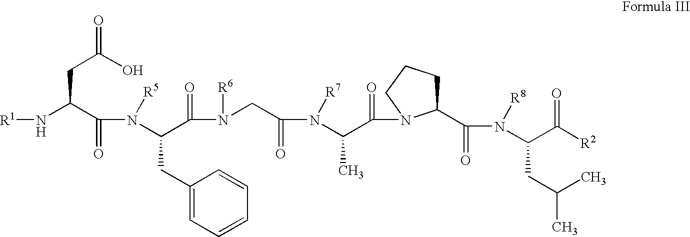
wherein R¹ is selected from H, optionally substituted C₂-C₆ acyl and optionally substituted C₁-C₆ alkyl, preferably H and acetyl and R² is selected from OH and NR³R⁴, wherein R³ and R⁴ are independently selected from H and optionally substituted C₁-C₆ alkyl preferably R² is selected from OH and NH₂; R⁵, R6, R⁷ and R⁸ are independently selected from H and C₁-C₆ alkyl.

In another embodiment, the invention provides compounds according to Formulae II or III, wherein R⁵, R⁶, R⁷ and R⁸ are H.

In another embodiment, the invention provides compounds according to Formulae II or III, wherein R¹ is H and R² is OH.

In another embodiment, the invention provides compounds according to Formulae II or III, wherein R¹ is acetyl.

In another embodiment, the invention provides compounds according to Formulae II or III, wherein R²is NH₂.

In another embodiment, the invention provides compounds according to Formula III wherein R¹ is acetyl and R²is NH₂.

In another embodiment of the invention, peptides of Formula I are selected from the group consisting of SEQ ID NO. 2, SEQ ID NO. 3 and. SEQ ID NO. 4.

In another embodiment of the invention, peptides of Formula I can be used for the preparation of a medicament for the treatment or prevention of IAPP related disorders such as diabetes Type I or Type II, e.g. to prevent or delay the aggregation of amylin associated with the onset and/or progression of Type II diabetes or for the treatment of islet cells, e.g. cultured pancreatic islet cells in vitro prior to their transplantation, for the treatment of Type I diabetes patients, e.g., post-transplantation, to prevent or inhibit fibril formation in the transplanted cells.

Still another embodiment of the present invention, is a method for treating or preventing an IAPP related amyloidosis, such as a diabetic disorder, preferably Type II diabetes or post-transplantation Type I diabetes.

A further embodiment of the invention is a method for treating or preventing IAPP disorders wherein the method comprises administering an effective dose of the above-mentioned peptides and derivatives thereof to a subject in the need thereof, wherein the subject can be human or animal, preferably human.

A further embodiment of the invention comprises the administration of at least a compound of the invention in a regimen coordinated with insulin or with glucose sensitizers, e.g. in a treatment for diabetes for simultaneous, sequential or separate use.

Pharmaceutical compositions comprising at least one peptide of the invention include all compositions wherein the peptide(s) are contained in an amount effective to achieve the intended purpose. In addition, the pharmaceutical compositions may contain suitable pharmaceutically acceptable carriers comprising excipients and auxiliaries which facilitate processing of the active compounds into preparations which can be used pharmaceutically. Suitable pharmaceutically acceptable vehicles are well known in the art and are described for example in Gennaro et al, 2000, a standard reference text in this field. Pharmaceutically acceptable vehicles can be routinely selected in accordance with the mode of administration, solubility and stability of the peptides. For example, formulations for intravenous administration may include sterile aqueous solutions which may also contain buffers, diluents and other suitable additives. The use of biomaterials and other polymers for drug delivery, as well the different techniques and models to validate a specific mode of administration, are disclosed in literature (Luo et al., 2001 and Cleland et al., 2001).

The above-mentioned peptides and derivatives of the present invention may be administered by any means that achieve the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, intranasal or buccal. Preferably the compounds of the invention are administered by subcutaneous, intramuscular or intravenous injection or infusion.

Parenteral administration can be by bolus injection or by gradual perfusion over time. A typical regimen for preventing, suppressing, or treating amylin misfolding related disorders, comprises either (1) administration of an effective amount in one or two doses of a high concentration of inhibitory peptides in the range of 0.5 to 10 mg of peptide, more preferably 0.5 to 5 mg of peptide, or (2) administration of an effective amount of the peptide in multiple doses of lower concentrations of inhibitor peptides in the range of 10-1000 μg, more preferably 50-500 μg over a period of time up to and including several months to several years. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

Preparations for parenteral administration include sterile aqueous or non-aqueous solutions, suspensions, and emulsions, which may contain auxiliary agents or excipients which are known in the art. Suitable formulations for parenteral administration include aqueous solutions of the active compounds in water-soluble form, for example, water-soluble salts. In addition, suspension of the active compound as appropriate oily injections suspensions may be administered.

Depending on the intended route of delivery, the compounds may be formulated as injectable or oral compositions. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a pre-determined quantity of active material calculated to produce the desired therapeutic effect, in association with a suitable pharmaceutical excipient. Typical unit dosage forms include pre-filled, pre-measured ampoules or syringes of the liquid compositions or pills, tablets, capsules, or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavours and the like. Solid forms may include, for example, any of the following ingredients, or compounds of a similar nature: a binder such as microcrystalline cellulose, gum tragacanth or gelatine; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavouring agent such as peppermint, methyl salicylata, or orange flavouring.

Injectable compositions are typically based upon injectable sterile saline or phosphate-buffered saline or other injectable carriers known in the art.

The above-described components for orally administered or injectable compositions are merely representative. Further materials as well as processing techniques and the like are known to the skilled practitioner (Gennaro et al., 2000).

The compounds of this invention can also be administered in sustained release forms or from sustained release drug delivery systems. A description of representative stained release materials is also known to the skilled practitioner (Karsa et al., 1993 and Yacobi et al., 1998).

By “effective amount”, is meant an amount sufficient to achieve a concentration of peptide(s) which is capable of slowing down or inhibiting the formation of amylin deposits, or of dissolving pre-formed deposits. Such concentrations can be routinely determined by those of skilled in the art. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like. It will also be appreciated by those of skilled in the art that the dosage may be dependent on the stability of the administered peptide. A less stable peptide may require administration in multiple doses.

The expression “Pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution.

Besides the pharmaceutically acceptable carrier, the compositions of the invention can also comprise minor amounts of additives, such as stabilizers, excipients, buffers and preservatives.

The present invention has been described with reference to the specific embodiments, but the content of the description comprises all modifications and substitutions, which can be brought by a person skilled in the art without ending beyond the meaning and purpose of the claims.

The invention will now be described by means of the following Examples, which should not be construed as in any way limiting the present invention. The Examples will refer to the Figures specified here below.

### BRIEF DESCRIPTION OF THE DRAWINGS

FIG. 1 compares sequences of human, cat, mouse, rat and hamster amylin. The amyloidogenic domain spanning residues 20-29 is shown by arrows and the sequence used as a template to generate β-sheet breaker peptides is highlighted in bold.

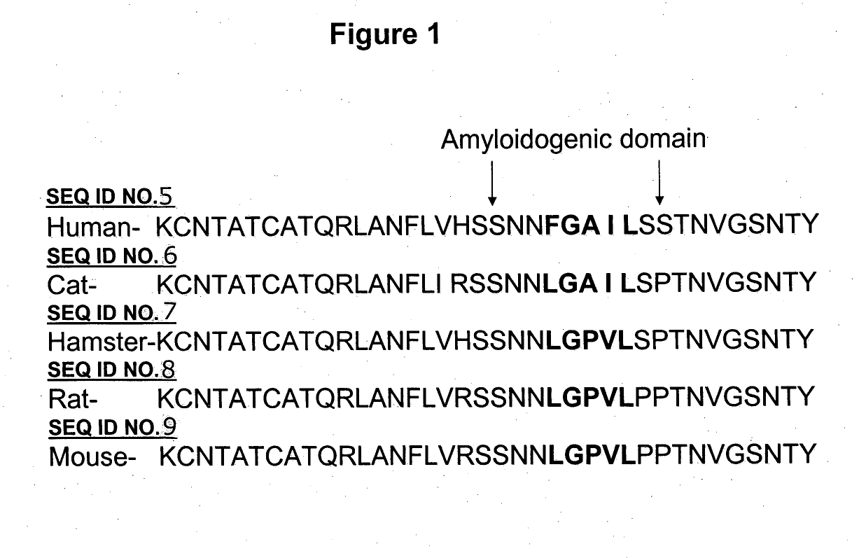

FIG. 2 shows the effect of peptide of Example SEQ ID NO. 2 on amyloid cytotoxicity. Soluble amylin (100 μM), soluble amylin (100 μM)+peptide of SEQ ID NO. 2 (800 μM), and peptide of SEQ ID NO. 2 (800 μM) were pre-incubated at 37° C. in Tris buffer pH 7.4 for 24 h then diluted in 50 μl of culture medium before addition to Rin-m5F cells, to yield final concentrations of 2 μM (amylin) and 16 μM (peptide of SEQ ID NO. 2). Data are expressed as average MTT reduction±SD (n=5) relative to cells treated with medium alone, which was made to equal 100%.

FIG. 3 shows the linear relationship between IC₅₀ of peptide of SEQ ID NO. 2 in preventing cytotoxicity in vitro and amylin concentration. Aliquots of 2.5, 10, 50 and 100 μM of amylin were incubated during 24 h at 37° C. with different concentrations of peptide of SEQ ID NO. 2. Thereafter, the samples were diluted in cell culture medium and cytotoxicity assay performed as described in Example 2.

Abbreviations

h (hour), μl (microliters), μM (micromolar), mg (milligrammes), min (minutes), ml (milliliters), mM (millimolar), MTT (3-4,5-dimethylthiazol-2-yl)-2,5-diphenyl terazolium bromide), nm (nanometers), PBS (Phosphate Buffered Sulfate), pM (picomolar), RT (room temperature), SDS (Sodium Dodecyl Sulfate, TFA (trifluoro acetic acid).

### EXAMPLES

The invention will be illustrated by means of the following examples which are not to be construed as limiting the scope of the invention.

The following examples illustrate preferred compounds according to Formula I, and methods for determining their biological activities.

Synthetic human amylin (1-37) (TFA salt) and Aβ₁₋₄₂ were synthesized in solid phase at W. M. KECK FOUNDATION, YALE UNIVERSITY. Amylin was not chemically reduced to make sure that the cystein bridge between amino acids 2 and 7 was formed. Control peptides, and the prion protein fragment 106-126 were purchased from NEOSYSTEM (Strausbourg). In order to increase the solubility of amylin (1-37), HCl salt of amylin by dissolving 1 mg of amylin in 1 ml of 2 mM HCl solution, sonicated for 1 min at RT in an ultrasonic water bath, then lyophilized in aliquots of 0.2 mg and kept dry at 4a° C. until use.

### Example 1

### Synthesis of Compounds of the Invention

Inhibitor peptides were synthesized in solid phase by NEOSYSTEM. Peptides were purified by HPLC and purity (>95%) evaluated by peptide sequencing and laser desorption mass spectrometry. Stock solution of the peptides were prepared in water/0.1% trifluoric acid and stored lyophilized in aliquots at −70° C. Concentration of the stock solution was estimated by amino acid analysis.

The chemical derivatization reactions were done during the synthesis by NEOSYSTEM using standard procedures.

The molecular weights measured by mass spectrometry are listed in Table I below:

TABLE I

| SEQ ID N^{o}. | MW (g/mol) |
|---|---|
| 2 | 619 |
| 3 | 660 |
| 4 | 734 |
| 10 | 720 |
| 11 | 705 |
| 12 | 749 |
| 13 | 699 |
| 14 | 790 |
| 15 | 776 |
| 16 | 733 |
| 17 | 618 |

### Example 2

### Biological Assays

In Vitro Peptide Solubility Assay:

Solubility of peptides of the invention was obtained using a qualitative assay where the peptide was dissolved in Tris buffer, pH 7.4 at 1, 5, or 10 mg/ml, vortexed briefly, and then centrifuged at 16,000 g for 30 min. The presence of a pellet in the bottom of the tube indicates that the peptide is not soluble at the respective concentration.

The data in Table II below indicate that peptides of the invention are highly soluble:

TABLE II

| SEQ ID N^{o}. | Solubilty | |
|---|---|---|
| 10 | <200 | μg/ml |
| 11 | <1 | mg/ml |
| 12 | 2 | mg/ml |
| 4 | >10 | mg/ml |
| 3 | >10 | mg/ml |
| 2 | >10 | mg/ml |

In Vitro Assays of Activity:

The activity of compounds of the invention in inhibiting the formation of aggregated amylin fibrils can be tested by absorbance changes.

Amyloid formation was quantitatively evaluated by measuring the amount of bound Congo red (Cb) to the fibrils using the formula below as reported (Klunk et al., 1999):
Cb (μM)=(A541/47,800)−(A403/68,300)−(A403/86,200)
wherein A₅₄₁ and A₄₀₃ are the absorbances respectively at 541 and 403 nm.

Aliquots of amylin at a concentration of 0.4 mg/ml prepared in 50 mM Tris-HCl, pH 7.4 were incubated at 37° C. for 48 h in the absence or presence of 1 mg/ml of the peptide of the invention. At the end of the incubation time, 125 μl of 15 μM Congo rod in PBS was added to 25 μl of sample, mixed and incubated for 10 min at RT. The absorbance of the resulting solutions was then measured at 403 and 541 nm. The amount of Congo red was calculated using the above formula. The amount of Congo red bound to amylin fibrils in the absence of the inhibitor was set to 100%.

The data presented in Table III below indicate that peptides of the invention inhibit the formation of amylin aggregates:

TABLE III

| SEQ ID N^{o}. | % Inhibition of Amylin fibrils |
|---|---|
| 5 (Amylin) | 0 |
| 12 | 20 |
| 13 | 16 |
| 14 | 8 |
| 15 | 10 |
| 16 | 24 |
| 4 | 39 |
| 3 | 47 |
| 2 | 43 |

Cellular Assays of Activity:

The mechanism proposed for the implication of amylin misfolding and aggregation in the pathogenesis of Type II diabetes is by inducing islet β-cell death and thus pancreas dysfunction. The ability of compounds of the invention to prevent or reduce the formation of cytotoxic amylin fibrils (Lorenzo et al., 1994) was evaluated by measuring the inhibition of amylin induced cytotoxicity in pancreatic β-cells.

Toxicity was measured by comparing the effects of amylin or amylin combined with peptides of the invention, on the reduction of the redox active dye, 3-(4,5-dimethylthiazol-2-yl)-2,5-diphenyltetrazolium bromide (MTT) by PC12 cells or Rin-m5F cells (ATCC), a pancreatic beta cell line. Cells were grown in RPMI-1640 media containing 10% foetal bovine serum, and plated onto 96-well plates.

Amylin at 0.2 mg/ml in 50 mM Tris-HCl pH 7.4, in the absence or presence of 1 mg/ml of peptide of the invention, was incubated at 37° C. for 48 h, then diluted in 50 μl of culture media at the appropriate peptide concentration before addition to the Rin-m5F cells. After overnight incubation, 10 μl of 2.5 mg/ml MTT was added to each well and the incubation continued for a further 3 h. Cells were then solubilized in 200 μl of 20% (w/v) SDS in 50% (v/v) N,N′-dimethylformamide, 25 mM HCl, 2% (v/v) glacial acetic acid, pH 4.7, by overnight incubation at 37° C. Levels of reduced MTT were determined by measuring the difference in absorbance at 595 and 650 nm using a microplate reader.

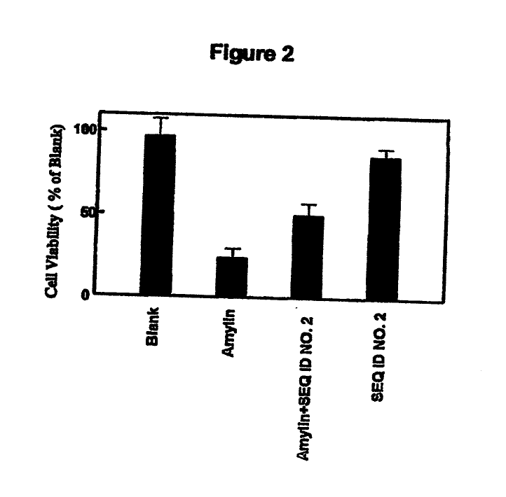

Cell viability is measured in presence of amylin alone and for a mixture of amylin and peptides of the invention. Cell viability in presence of a mixture of amylin and compound of SEQ ID NO. 2 is presented in FIG. 2 in comparison with amylin alone. The percentage of inhibition of cellular toxicity is calculated for the mixtures in comparison to cellular toxicity induced by amylin alone. Percentages of inhibition of cellular toxicity are presented in Table IV below for compounds of the invention:

TABLE IV

| | % Inhibition |
|---|---|
| SEQ ID N^{o}. | Amylin fibrils cytotoxicity |
| 5 (Amylin) | 0 |
| 17 | 19 |
| 4 | 33 |
| 3 | 43 |
| 2 | 40 |

Since the concentration at which the inhibitory effect of peptides of the invention is observed depends on the concentration of amylin used, the concentration of amylin was varied and the inhibitory concentration at 50% of the effect (IC₅₀) of compound of SEQ ID NO. 2 was calculated. For this, aliquots of 2.5, 10, 50 and 100 μM of amylin were incubated during 24 h at 37° C. with different concentrations of peptide SEQ ID NO. 2. Thereafter, the samples were diluted in cell culture medium and cytotoxicity assay performed.

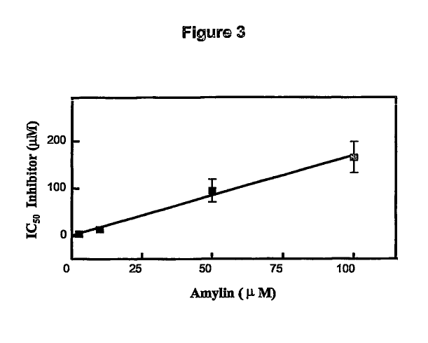

As shown in FIG. 3. the IC₅₀ for the peptide of SEQ ID NO. 2 has a linear relationship with amylin concentration. IC₅₀ were found to be 1.5-2.7-fold higher than amylin concentrations used in the study. Considering that amylin concentration in blood has been shown to be 2-3 μM, we estimate that the peptide concentration in plasma in which a 50% of activity would be reached in vivo is 4-6 pM.

Selectivity of the effect of peptides of the invention was assayed in the same assay as presented above.

Selectivity of peptides of the invention towards amylin was tested by measuring the ability of peptides of the invention to inhibit cellular oxicity induced by prion protein fragment (PrP₁₀₆₋₁₂₆) by Aβ₁₋₄₂ peptide. Peptides of the invention were not able to inhibit either cellar toxicity of prion, nor Aβ fibrils.

### REFERENCES

Angainao et al. 2002, Biochemistry, 41, 11338-11343;
Carrell et al. 1997, The Lancet, 350, 134-8;
Cleland et al., Curr. Opin. Biotechnol., 12:212-9, 2001;
Cooper G J et al. 1987, Proc. Nad. Acad. Sci. U.S.A., 84:8628-32;
Dobson 1999, Trends Biochem. Sci. 24, 329-32;
Fezoul et al. 1999, Int J. Exp. Clin. Invest. 7, 166-178;
Gennaro et al. 2000, of Remington's Pharmaceutical Sciences, Part 8, 20^{th} Edition, Marck Publishing Company, Easton, Pa.;
Glenner et al. 1988, Biochem. Biophys. Res. Commun 155, 608;
Höppener J W M et al. 2000, N. Engl. J. Med, 343, 411-419;
Karsa et al. 1993, (Ed) Encapsulation and Controlled Release; Stephenson (Editor); Springer Verlag;
Klunk et al. 1999, Methods Enzymol., 309:285-305;
Lorenzo et al. 1994, Nature 368: 756-760;
Luo et al. 2001, Exp. Opin. Ther. Patents, 11: 1395-1410;
Moriarty at al. 1999, Biochemistry, 38, 1811-1818;
Rink et al. 1993, Trends in Pharmacol. Sci. 14, 113-118;
Soto et al. 2000, FEBS Lett., 498, 204-7;
Westermark et al. 1990, Proc. Natl. Acad. Sci. U.S.A 87:5036-40;
Yacobi et al. 1998, Oral Sustained Release Formulations: Design and Evaluation, Eva Halperin-Walega (Editor), 1st Ed. edition; Pergamon Press;
WO 96/39834, New York University;
WO 01/34631, Axonyx Inc;
EP 885904 Fraucnhofer Ges. Forshung;
US 2002/0119926;
EP 1162207 Amylin Pharmaceuticals;
U.S. Pat. No. 6,610,824 Amylin Pharmaceuticals.

1. A peptide having an amino acid sequence of Formula I (SEQ ID NO: 1):
X₁FGAPX₂ X₃ in which
X₁, is selected from Aspartic acid and a derivative thereof selected from acylated and alkylated Aspartic acid;
X₂ is Leucine or when X₃ is absent, X₂ is selected from Leucine and amidated Leucine;
X₃ is absent or selected from Aspartic acid and amidated Aspartic acid;
as well as salt and any derivative or analogue thereof.

2. A peptide according to claim 1 wherein X₁ and X₃ are Aspartic acid.

3. A peptide according to claim 1 wherein X₃ is absent.

4. A peptide according to claim 1 of the Formula II below: 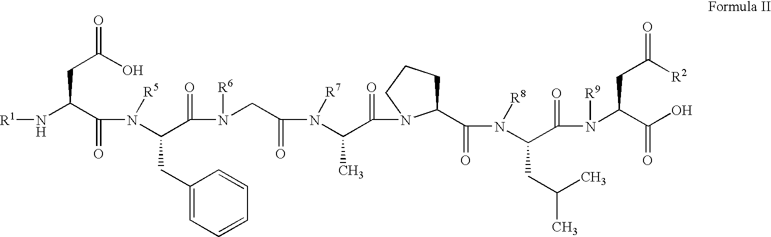
wherein R¹ is selected from H, C₂-C₆ acyl and C₁-C₆ alkyl; R² is selected from OH and NR³R⁴, wherein R³ and R⁴ are independently selected from H and C₁-C₆ alkyl and R⁵, R⁶R⁷, R⁸ and R⁹ are independently selected from H and C₁-C₆ alkyl.

5. A peptide according to claim 4 wherein R¹ is selected from H and C₂-C₆ acyl and R² is selected from OH and NH₂.

6. A peptide according to claim 4 wherein R¹ is H and R² is OH.

7. A peptide according to claim 1 of the Formula III below: 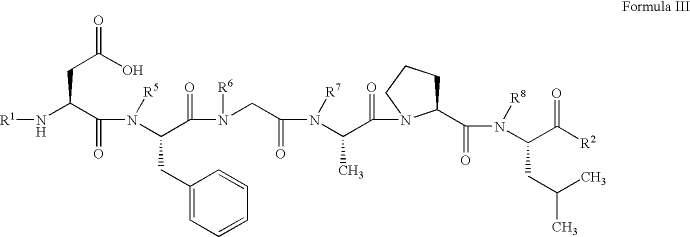
wherein R′ is selected from H, C₂-C₆ acyl and C₁-C₆ alkyl; R² is selected from OH and NR³R⁴, wherein R³ and R⁴ are independently selected from H and C₁-C₆ alkyl and R⁵, R⁶, R⁷and R8 are independently selected from H and C₁-C₆ alkyl.

8. A peptide according to claim 7 wherein R¹ is selected from H and C₂-C₆ acyl and R² is selected from OH and NH₂.

9. A peptide according to claim 7 wherein R¹ is acetyl and R² is NH₂.

10. A peptide according claim 4 wherein R⁵, R⁶, R⁷and R⁸ are H.

11. A peptide according claim 1 selected from the following group:
SEQ ID NO. 2, SEQ ID NO. 3 and SEQ ID NO. 4.

12. A compound comprising the peptide according to claim 1 for use as a medicament.

13. A method for the preparation of a medicament for the treatment or prevention of an amyloidosis disorder related to IAPP comprising adding a peptide according to claim 1 to the medicament formulation.

14. The method according to claim 13 wherein the amyloidosis disorder is a diabetic disorder.

15. The method according to claim 14 wherein the diabetic condition is Type II diabetes.

16. The method according to claim 14 wherein the diabetic condition is post-transplantation Type I diabetes.

17. A pharmaceutical composition comprising a the peptide according to claim 1 as active ingredient and a pharmaceutically acceptable excipient or carrier.

18. A method of treating or preventing diabetic disorders by administering an effective amount of the peptide according to claim 1 to a subject in the need thereof.

19. A method according to claim 18, in which the subject is human.

20. A peptide according to claim 7 wherein R⁵, R⁶, R⁷and R⁸ are H.


SEQUENCE LISTING (19 sequences)

SEQ ID NO: 1 - protein, 7 aa, Artificial Sequence
  Synthetic Peptide
  misc_feature (1)..(1): Xaa can be any naturally occurring amino acid
  misc_feature (6)..(7): Xaa can be any naturally occurring amino acid
XFGAPXX

SEQ ID NO: 2 - protein, 6 aa, Artificial  Sequence
  Synthetic Peptide
DFGAPL

SEQ ID NO: 3 - protein, 6 aa, Artificial Sequence
  Synthetic Peptide
  misc_feature (1)..(1): Xaa can be any naturally occurring amino acid
  misc_feature (6)..(6): Xaa can be any naturally occurring amino acid
XFGAPX

SEQ ID NO: 4 - protein, 7 aa, Artificial Sequence
  Synthetic Peptide
DFGAPLD

SEQ ID NO: 5 - protein, 37 aa, Homo sapiens [sequence omitted]

SEQ ID NO: 6 - protein, 37 aa, Felis catus
KCNTATCATQRLANFLIRSSNNLGAILSPTNVGSNTY

SEQ ID NO: 7 - protein, 37 aa, Mesocricetus auratus
KCNTATCATQRLANFLVHSSNNLGPVLSPTNVGSNTY

SEQ ID NO: 8 - protein, 37 aa, Rattus norvegicus
KCNTATCATQRLANFLVRSSNNLGPVLPPTNVGSNTY

SEQ ID NO: 9 - protein, 37 aa, Mus musculus; identical to SEQ ID NO: 8

SEQ ID NO: 10 - protein, 7 aa, Homo sapiens
NFGAILS

SEQ ID NO: 11 - protein, 7 aa, Artificial Sequence
  Synthetic Peptide
NFGAPLS

SEQ ID NO: 12 - protein, 7 aa, Artificial Sequence
  Synthetic Peptide
DFGAILD

SEQ ID NO: 13 - protein, 7 aa, Artificial Sequence
  Synthetic Peptide
DPGAILD

SEQ ID NO: 14 - protein, 7 aa, Artificial Sequence
  Synthetic Peptide
DFPAILD

SEQ ID NO: 15 - protein, 7 aa, Artificial Sequence
  Synthetic Peptide
DFGPILD

SEQ ID NO: 16 - protein, 7 aa, Artificial Sequence
  Synthetic Peptide
DFGAIPD

SEQ ID NO: 17 - protein, 6 aa, Artificial Sequence
  Synthetic Peptide
FGAPLD

SEQ ID NO: 18 - protein, 5 aa, Artificial
  Synthetic Peptide
LPFFD

SEQ ID NO: 19 - protein, 5 aa, Artificial
  Synthetic Peptide
  MOD_RES (1)..(1): Acetylated Nitrogen
  MOD_RES (5)..(5): Amidated Carboxyl Group
LPFFD